(19) United States
(12) Patent Application Publication
(10) Pub. No.: US 2025/0114233 A1
(43) Pub. Date: Apr. 10, 2025

(54) DEVICE FOR DRAINING URINE BY MEANS OF A NON-ADHESIVE ATTACHMENT TO THE PENIS

(71) Applicants: UNIVERSITE DE MONTPELLIER, MONTPELLIER (FR); CENTRE HOSPITALIER UNIVERSITAIRE DE MONTPELLIER, MONTPELLIER CEDEX 5 (FR); ECOLE NATIONALE SUPERIEURE DE CHIMIE DE MONTPELLIER, MONTPELLIER CEDEX 5 (FR); CENTRE NATIONAL DE LA RECHERCHE SCIENTIFIQUE, PARIS (FR)

(72) Inventors: Xavier GARRIC, MONTPELLIER (FR); Benjamin NOTTELET, MONTPELLIER (FR); Rodolphe THURET, MONTPELLIER (FR); Coline PINESE, SAINT VALLIER (FR)

(73) Assignees: UNIVERSITE DE MONTPELLIER, MONTPELLIER (FR); CENTRE HOSPITALIER UNIVERSITAIRE DE MONTPELLIER, MONTPELLIER CEDEX 5 (FR); ECOLE NATIONALE SUPERIEURE DE CHIMIE DE MONTPELLIER, MONTPELLIER CEDEX 5 (FR); CENTRE NATIONAL DE LA RECHERCHE SCIENTIFIQUE, PARIS (FR)

(21) Appl. No.: 18/729,999

(22) PCT Filed: Jan. 20, 2023
(86) PCT No.: PCT/FR2023/050086

§ 371 Date: Jul. 18, 2024

(30) Foreign Application Priority Data
Jan. 21, 2022 (FR) FR2200527

(51) Int. Cl. A61F 5/453

(52) U.S. Cl. CPC A61F 5/453

(57) ABSTRACT

The field of devices for the draining urine that are attached to the penis of a patient, in particular a patient with urinary incontinence, without a medical glue or adhesive. The device includes a balanic sheath and an attachment system, wherein the balanic sheath includes a proximal portion, the proximal portion of the balanic sheath having a frustoconical inner surface and an outer surface each having a distal end having a small diameter and a proximal end having a large diameter, wherein the attachment system is configured to hold the retracted foreskin on the outer surface of the proximal portion, the balanic sheath including a distal portion extending the proximal portion with an outer surface having a protrusion, such that the retracted foreskin is held by the attachment system between the proximal portion and the distal portion.

### FIELD

The present disclosure relates to the field of urine drainage devices to fasten to the penis of a patient, for example a patient suffering from urinary incontinence, without adhesive or medical glue.

### BACKGROUND

Urinary incontinence is characterized by an involuntary loss of urine through the urethra. Several types of urinary incontinence exist according to their pathophysiological cause. Whatever the cause, however, the repercussions on the daily life of the patient are numerous and can lead to great social discomfort.

A number of therapeutic solutions, which may or may not involve medication, are available for improving the lives of patients suffering from urinary incontinence.

Among the solutions not involving medication, a distinction is made in particular between non-absorbent devices and absorbent devices such as diapers. Non-absorbent devices notably comprise:
adhesive or non-adhesive penis sheaths, external urine collection devices. A penis sheath conventionally has a shape similar to that of a condom and it generally comprises an assembly composed of the sheath, placed on the penis, and an endpiece to which tubing is attached, the tubing being connected to a collection bag;
urinary catheters, tubes introduced into the urinary meatus up to the bladder, intended to collect urine.

Although urinary catheters have proven effective, they are unpleasant and painful for the patient and may be an obstacle to certain everyday movements. They can also be sources of infection.

Urine drainage devices which comprise penis sheaths using an adhesive or medical glue to fasten the sheath to the penis or to the glans of the patient's penis have several drawbacks. The adhesive tapes attached to the fine skin of the patient's penis, or directly to the glans of the patient's penis, are sources of irritation. Removing the sheath can be painful, and fitting it in place can be tedious since it is difficult to adjust the position of the sheath on the penis once the sheath is stuck to the skin. In addition, it is sometimes recommended to leave the penis sheath in place for several days; the quality of the adhesive may then deteriorate, and the fixation of the sheath is no longer guaranteed.

To overcome these problems, penile sheaths have been provided that fasten mechanically to the patient's penis by “pinching”, “tightening” or in an equivalent manner “exerting pressure” on the patient's foreskin.

The American patent U.S. Pat. No. 4,388,923 describes a urine drainage device in the form of a condom which unrolls only on the glans of the patient's penis. Once the device has been placed on the glans, the patient unrolls his foreskin over the device.

The device also comprises an elastic sheath that can unroll from one side or the other of the condom, in the manner of an umbrella that folds up in one direction or another. Thus, the sheath can be unrolled on the foreskin so as to enclose the latter and hold the device tight on the patient's foreskin. The device ends in a tubing that concentrates the patient's urine into a bag.

Other patent applications disclose improvements to the device of U.S. Pat. No. 4,388,923. For example, the patent application US 2001/0005782 describes placing an elastic connection between the tubing and the end of the condom placed on the glans. Thus, when the bag is filled with urine and could risk pulling the end of the device downward by gravity and therefore “tightening” the foreskin more tightly, the elastic band relieves the device from the weight of the bag and prevents the patient's foreskin from being tightened too tightly. The patent application US 2004/0087921 discloses that the membrane covering the foreskin is made of a breathable material allowing gas exchange but not liquid exchange.

The devices described above have the following drawbacks: they are difficult to adapt to different morphologies of the glans and penises of patients. Indeed, the dimension of the device is limited by the size of the elastic sheath which unrolls over the foreskin. The elasticity of the sheath decreases over time, and the fixation of the device is no longer ensured. The patient cannot adjust the tightness of the device. Finally, the sheath abruptly retracts under an elastic effect upon removal of the device, and splashes unpleasant for the patient may occur.

The device of the present invention is a urine evacuation device that would improve the situation and solve the problems mentioned above. Moreover, the device of the present invention can be used by subjects, for example aviators or glider pilots, who do not necessarily suffer from urinary incontinence but who need a urine evacuation device because they do not have access to toilets for a long period of time.

### SUMMARY

According to a first aspect, the invention is a urine drainage device comprising a non-adhesive balanic sheath (balanic is an adjective meaning “relating to the glans of the penis”) and a fastening system,
wherein the balanic sheath comprises a proximal part for receiving the exposed glans of a patient, the proximal part of the balanic sheath having a frustoconical inner surface and an outer surface, the inner surface and the outer surface of the proximal part of the sheath each comprising a distal end with a small diameter and a proximal end with a large diameter,
wherein the fastening system is configured to hold the patient's foreskin unrolled on the outer surface of the proximal part of the balanic sheath,
characterized in that the balanic sheath comprises a distal part continuing the proximal part, with an outer surface having a protuberance such that the foreskin, when it is unrolled, is held by the fastening system in a zone straddling the proximal part and the distal part, upstream of the protuberance.

In the remainder of the description, “sheath” and “balanic sheath” will be used as equivalents to designate the same shape.

The particular shape of the balanic sheath, combined with the protuberance, makes it possible to effectively hold the balanic sheath in place on the patient's penis by means of the fastening system which tightens the foreskin upstream of the protuberance, straddling the proximal and distal parts of the balanic sheath. The specific position and shape of the protuberance allow easy placement of the sheath on the penis. The protuberance also allows effective tightening of the fastening system on the sheath.

According to one embodiment of the present invention, the protuberance is a continuous or non-continuous circular bead, provided on a disk perpendicularly intersecting the distal part of the balanic sheath, when the latter is viewed from the side with the axis of the frustoconical proximal part collinear with a vertical axis.

Placing a continuous or non-continuous bead as a protuberance creates a continuous or non-continuous hollow on the outer surface of the sheath, straddling the proximal part and the distal part of the sheath, and makes it possible to hold the foreskin effectively in place while preventing the fastening system from slipping.

According to one embodiment of the present invention, the fastening system is a non-rigid tightening collar. The non-rigid collar is preferably adaptable to the thickness and length of the foreskin. The non-rigid tightening collar is, for example, a touch-and-close band (which may or may not be elastic), or a cord which is tightened by mean of a tightening button of the cord lock type, or a cord (in particular elastic) which is closed by mean of a pushbutton, or an elastic circular band, or a flexible magnet in the form of a band, or a cord (in particular elastic) which closes by mean of a magnet.

During the day, the size of the penis may vary and the patient may need to adapt the fixation of the device on the penis. The use of a non-rigid and adaptable tightening collar allows the patient to very easily adjust the tightening of the foreskin on the outer surface of the balanic sheath.

According to another embodiment of the present invention, the fastening system is a rigid tightening collar. The rigid collar can comprise two articulated parts which are closed around the foreskin, for example by mean of a touch-and-close fastener, a magnetic fastener or a pin-type closure system. A pin-type closure system comprises a first part with at least one hole that receives the pin of a second part of the system. This type of closure is conventionally used for tightening and closing a watch strap.

According to another embodiment, the fastening system is a clip system, of which the clips tighten the foreskin on the balanic sheath upstream of the protuberance, straddling the proximal part and the distal part of the sheath, and the operating mechanism of the clips is located downstream of the protuberance, at the distal part of the sheath. The operating mechanism of the clips is, for example, a spring-operated system. Otherwise, the end of the clips coming into contact with the foreskin in order to tighten it onto the sheath can comprise a magnet which fastens magnetically to another magnet provided in or on the sheath. Moreover, the operating mechanism of the clips can be a ring which is to be screwed onto a thread provided on the distal part of the sheath or clipped into a notch provided on the distal part of the sheath such that, when the ring is screwed or clipped on downstream of the protuberance, the clips tighten onto the patient's foreskin upstream of the protuberance. The clips can also be held around the foreskin by surrounding them and tightening them by mean of a non-rigid tightening collar as described above.

The clip system allows the patient to more conveniently fasten the balanic sheath to the patient's foreskin and to remove the sheath simply by loosening the clips. Moreover, the clip system can be modified in order to tighten the clips more or less firmly on the foreskin, for example by adjusting the elastic force of the springs.

According to another embodiment, the fastening system is a fastening system of the “skirt” type comprising a first circular elastic band downstream of the protuberance and a non-rigid tightening collar, as described above, placed so as to tighten the patient's foreskin upstream of the protuberance, straddling the distal part and the proximal part of the balanic sheath. The first elastic band and the non-rigid tightening collar are connected to each other by a fine textile such as silk or cotton or a silicone weave.

The fastening system of the “skirt” type ensures the fixation of the foreskin on the balanic sheath on the one hand and the fixation of the fastening system on the sheath on the other hand.

The inner surface of the balanic sheath can be made of a different material than the outer surface thereof.

The possibility can be given of changing the materials of the sheath so that the contact between the inner surface of the sheath and the glans is as comfortable as possible for the user while at the same time minimizing irritation.

The balanic sheath can also be made in one piece from a single material.

The balanic sheath can be made, for example, of a material or a combination of materials chosen from the families of polyolefins, styrenes, vinyls, polyamides, polyesters, polyketides, polysulfides, polysulfones, polyacetals, thermoplastic elastomers, polycarbonates, fluorinated polymers. Preferably, the material is chosen from thermoplastic polymers and silicones. More preferably, the material is chosen from polyvinyl chloride, silicones, polyethylene, polystyrene, polypropylene, polyethylene terephthalate, polyacrylate and polymethyl acrylate and polyurethane.

Examples of materials from the families mentioned above are given according to their acronyms used in the reference works; the scientific names corresponding to these acronyms are therefore given in English.

For example, the family of polyolefins includes LDPE (low density polyethylene), HDPE (high density polyethylene), LLDPE (linear low-density polyethylene), PP (polypropylene), EVA (ethylene vinyl acetate).

For example, the family of styrenes includes PS (polystyrene), SAN (styrene acrylonitrile), ABS (acrylonitrile butadiene styrene), HIPS (high impact polystyrene).

For example, the vinyl family includes PVC (poly(vinyl chloride)), PVDC (PVDC).

For example, the family of polyamides includes nylon 6, 6/6, 6/10, 6/12, 11, 12.

For example, the family of polyesters includes PET (polyethylene terephthalate), PBT (poly(butylene terephthalate)), PETG (polyethylene terephthalate glycol) and PLA (polylactides), PGA (polyglycolides), PLGA (poly(lactide-coglycolides), PCL (polycaprolactone), PBL (poly-butyrolactone), PHA (polyhydroxyalkanoates), and their copolymers.

For example, the polyketide family includes PEEK (polyetheretherketone).

For example, the polysulfide family includes PPS (polyphenylene sulfide).

For example, the polysulfone family includes PSU, PES (polyethersulfone).

For example, the family of polyacetals includes POM (polyoxymethylene).

For example, the family of thermoplastic elastomers includes PU (polyurethane), PEBA (polyether block amide), COPE (polyether-ester block copolymer thermoplastic elastomers (COPE/TEEE)), TPOs (blend of polyolefins), EPDM (ethylene propylene diene modified rubber), EPR (ethylene propylene rubber), FPO (flexible polyolefin), POE (polyolefin elastomer), SBC (styrenic block copolymers), SBS (styrene-butadiene-styrene), SEBS (styrene-ethylene-butylene-styrene) and SIS (styrene-isoprene-styrene), TPV (thermoplastic vulcanizate), TPU (thermoplastic polyurethane), segments of flexible blocks of polyether or polyester coupled to hard blocks of aromatic or aliphatic isocyanate, COPE (copolyester), PEBA (elastomer of polyether block amide and copolyamide).

For example, the family of polycarbonates includes polycarbonate.

For example, the family of fluorine polymers includes PVDF (polyvinylidene fluoride), ETFE (ethylene-tetrafluoroethylene), PTFE (polytetrafluoroethylene).

The balanic sheath, like the fastening system, can be washable and sterilizable. Thus, the device can be reused by the user.

The length of the proximal part of the sheath can be between 5 and 25 mm, preferably between 13 and 20 mm, and more preferably 17 mm.

The ratio between the large diameter of the inner surface of the proximal part of the sheath and the smallest diameter of the distal end of the proximal part of the sheath can be between 1 and 3, more preferably 2.

Such a ratio allows easy placement of the balanic sheath onto the glans of the penis, as well as an optimal flow of urine, so that there is no risk of a drop of urine stagnating at the glans of the patient's penis. Thus, risks of infections are avoided.

The protuberance can be in the shape of a half-ball or a section of a ball. When the sheath is seen from the side with its longitudinal axis collinear with a vertical axis, the protuberance, seen from the side, is then a semicircle or an arc of a circle forming the outer surface of the distal part of the sheath at the protuberance, characterized by a diameter in the case where the protuberance is a half-ball or by an arc length in the case of a ball section, and a thickness which is the maximum distance between the inner surface and the outer surface of the distal part of the sheath at the protuberance, and the distance between the center of the half-ball and the proximal end of large diameter of the proximal part of the sheath.

The diameter or arc length can be between 5 and 15 mm, preferably between 7 and 16 mm, and more preferably 9 mm. The thickness can be between 3 and 20 mm, preferably between 5 and 15 mm, and more preferably 7 mm. In the case where the protuberance is a half-ball, the thickness of the protuberance is in fact equal to the diameter of the ball. In the case where the protuberance is a ball section, seen from the side, the thickness of the protuberance is the chord height, which is preferably smaller than the diameter of the ball. The distance between the center of the half-ball and the proximal end of large diameter can be between 15 and 25 mm and preferably 21 mm.

In the case where the protuberance is a bead, the bead, seen from the side when the longitudinal axis of the balanic sheath is collinear with a vertical axis, can have a shape according to a bell curve or Gaussian curve, the bead can be characterized by a height of between 5 and 20 mm, preferably of between 5 and 15 mm, and more preferably 9 mm. The thickness of the bead between the inner surface and the outer surface can be between 3 and 20 mm, preferably between 5 and 15 mm, and more preferably 7 mm.

The shape of the bead is therefore a little more distinctive than that of a half-ball. Moreover, the bead goes round the sheath continuously or non-continuously, while the protuberance in the form of a half-ball or of a ball section is only a “hump” which appears on one side of the sheath.

These dimensions make it possible to ensure an effective fixation of the fastening system on the device without the risk of the device “slipping” on the foreskin.

According to another aspect, the invention relates to a kit comprising the urine drainage device of the present invention, tubing, and a urine collection bag.

The balanic sheath of the urine drainage device can be produced, for example, by 3D printing, injection or extrusion.

### BRIEF DESCRIPTION OF THE DRAWINGS

Other features, details and advantages will appear on reading the detailed description below, and on analyzing the appended drawings, in which the protuberance is illustrated as a bead.

FIG. 1 shows a side view of the balanic sheath when its longitudinal axis is collinear with a vertical axis, according to one embodiment.

FIG. 2 shows a sectional view of the balanic sheath when its longitudinal axis is collinear with a vertical axis, according to one embodiment.

FIG. 3 shows a perspective view of the balanic sheath according to one embodiment of the present invention.

FIG. 4 shows a front view, through the distal end of the distal part of the balanic sheath of the urine drainage device according to an embodiment of the present invention, where the fastening system is a non-rigid tightening collar in the form of a cord which is tightened using a tightening button of the cord lock type.

FIG. 5 shows a side view of the urine drainage device when the longitudinal axis of the balanic sheath is collinear with a vertical axis, according to an embodiment where the fastening system is a rigid tightening collar comprising two articulated parts which are closed around the foreskin by mean of a pin-type fastening system.

FIG. 6 shows a side view of the device when the longitudinal axis of the balanic sheath is collinear with a vertical axis, according to an embodiment where the fastening system is a clip system, of which the clips tighten the foreskin on the balanic sheath upstream of the bead and the clip operating mechanism is located downstream of the bead, the fastening system being open.

FIG. 7 shows a process diagram in which the clip operating mechanism is a ring which screws onto a thread provided downstream of the bead on the distal part of the balanic sheath, such that the clips tighten onto the patient's foreskin upstream of the bead when the ring is screwed on.

FIG. 8 shows a side view of the urine drainage device when the longitudinal axis of the balanic sheath is collinear with a vertical axis, according to an embodiment where the fastening system is a clip system, of which the clips tighten the foreskin on the sheath straddling the proximal part and the distal part, and the clip operating mechanism is located downstream of the bead, the fastening system being tightened on the foreskin by means of a non-rigid tightening collar.

FIG. 9 shows a side view of the urine drainage device when the longitudinal axis of the balanic sheath is collinear with a vertical axis, according to an embodiment where the fastening system is open and the fastening system is of the “skirt” type.

FIG. 10 shows the side view with the fastening system is closed.

FIG. 11 shows, at the top, a side view and, at the bottom, a top view of the urine drainage device when the longitudinal axis of the balanic sheath is collinear with the horizontal, and of which the fastening system comprises a tightening band coupled to a lever fixed in rotation on the distal part of the balanic sheath. In this figure, the lever is in the open position.

FIG. 12 shows the same side view and the same top view as FIG. 11, with the lever in the closed position.

FIG. 13 shows the same side view and the same top view as FIG. 11, with the tightening band tightening around the balanic sheath in the zone straddling the proximal part and the distal part.

### DETAILED DESCRIPTION

The urine drainage device of the present invention comprises a non-adhesive balanic sheath 10. In the present application, “non-adhesive” is understood to mean that the balanic sheath is not attached to the patient's penis or glans by mean of adhesive or medical glue.

Since the balanic sheath 10 of the urine drainage device of the present invention uses neither adhesive nor medical glue, the inventors had to produce a balanic sheath 10 having a specific shape and dimensions allowing the sheath to be held optimally on the patient's penis. To this aim, the inventors have devised a clever urine drainage device comprising a balanic sheath 10 and a fastening system which makes it possible to tighten the patient's foreskin on the sheath.

The balanic Sheath

The balanic sheath 10 of the urine drainage device comprises a proximal part 11 and a distal part 12. The proximal part 11 of the balanic sheath 10 is intended to be placed on the glans of the patient's penis. The distal part 12 of the balanic sheath 10 continues the proximal part 11. The distal part 12 of the balanic sheath is in material continuity with the proximal part 11 and is intended to collect and evacuate the urine, for example toward tubing connected to a bag. Thus, the distal part 12 ends in a connection part 13 for attachment to such tubing.

The balanic sheath 10 has a longitudinal axis which is an axis collinear with the height of the truncated cone of the inner surface of the proximal part 11 of the balanic sheath 10. In the remainder of the application, the “vertical axis” will be understood to mean an axis pointing upward, perpendicular to the ground. Thus, when the proximal end of large diameter of the balanic sheath 10 rests on a flat surface, the longitudinal axis of the balanic sheath 10 is collinear with a vertical axis.

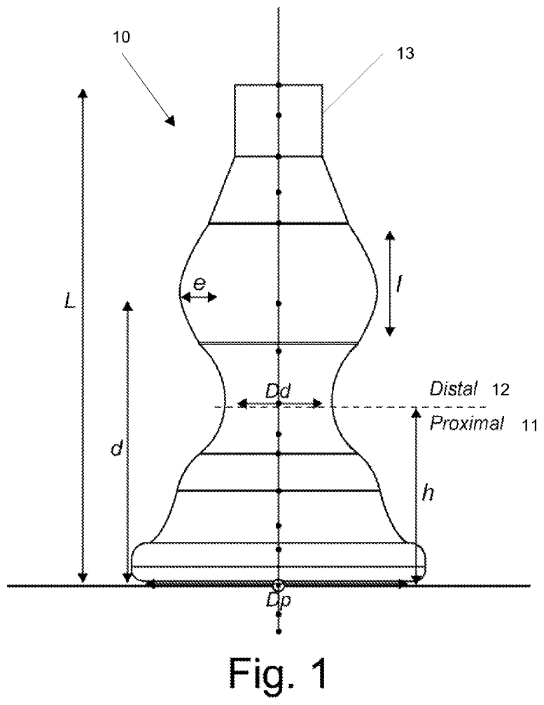

Reference is made to FIG. 1 which shows a side view of the balanic sheath 10 when its longitudinal axis is collinear with a vertical axis. The balanic sheath 10 can be characterized by different lengths. The total length L of the balanic sheath (proximal part plus distal part), considered along its longitudinal axis, can be between 15 and 60 mm, preferably between 30 and 40 mm, more preferably 35 mm.

However, in the case where the balanic sheath 10 has a tubing in material continuity with the distal part 12 of the sheath, in particular by fastening to the connection part 13, the total length L of the sheath is much greater, for example up to 1 m.

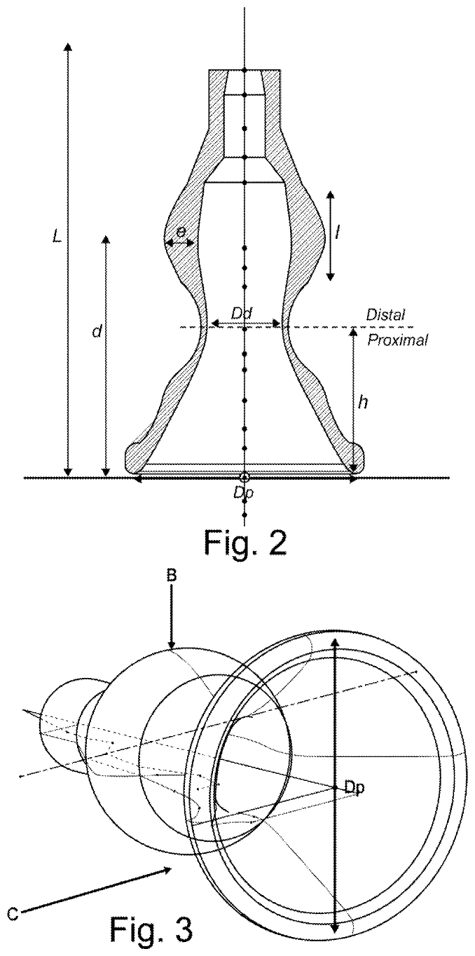

Reference is made to FIG. 2 which illustrates a balanic sheath 10 comprising a proximal part 11 and a distal part 12 that are delimited by a dashed line in the figure. The proximal part 11 comprises two surfaces, where the inner surface is of frustoconical shape and the outer surface forms slight ripples. The inner surface of the distal part 12 has an advantageous inner surface with several narrowed zones. In particular, the inner surface of the distal part 12 has, just after the proximal part 11 of the sheath, a zone of almost constant width which forms a reservoir, and then a zone which narrows to evacuate the urine from the reservoir.

Between its inner surface and its outer surface, the balanic sheath 10 therefore has a thickness delimited by the evolution of the inner surface on the one hand and the evolution of the outer surface on the other hand. The thickness of the sheath 10 is therefore not necessarily constant. As the inner surface follows a more or less complex shape, the outer surface of the sheath 10 also follows a certain shape. For example, the thickness of the sheath 10 can vary so as to create small waves at least in part, for example upstream of the bead B. Moreover, the thickness of the sheath 10 can be constant except at the protuberance.

The proximal part 11 can be made of a material different than that of the distal part 12 or of the same material.

### Proximal Part

When the patient places the inner surface of the proximal part 11 of the balanic sheath 10 on his glans, he must first of all have rolled his foreskin toward his pubic region. Once the proximal part 11 of the sheath 10 is placed on his glans, the patient unrolls his foreskin over the outer surface of the proximal part 11 of the sheath 10.

The inner surface of the proximal part 11 of the sheath 10 has a frustoconical shape characterized by a large diameter Dp, a small diameter Dd at its distal end, and a height h. The large diameter Dp is the proximal end of the proximal part 11 of the sheath, while the small diameter Dd is here the distal end of the proximal part 11 of the sheath. The frustoconical shape can be adapted to a large number of glans shapes and can be easily adjusted to best suit the patient's morphology.

The large diameter Dp can be between 7 and 25 mm, preferably between 10 and 20 mm, more preferably 20 mm.

The small diameter Dd can be between 5 and 10 mm, preferably 7 mm.

The height h of the truncated cone forming the proximal part 11 of the sheath can be between 5 and 25 mm, preferably between 13 and 20 mm, and more preferably 17 mm.

The ratio between the largest diameter Dp and the smallest diameter Dd of the truncated cone of the proximal part 11 of the sheath can be between 1 and 3, more preferably 2.

The outer surface of the proximal part 11 of the balanic sheath can be of the same shape as the inner surface of the proximal part 11 of the sheath, in which case the thickness between these two surfaces is constant.

Otherwise, the thickness between the two surfaces can vary. In this case, the outer surface of the proximal part 11 of the sheath can have variations, for example in the form of ripples. On the other hand, the outer surface, like the inner surface, comprises a large diameter at the proximal end of the proximal part 11 and a small diameter at the distal end of the proximal part 11 of the drainage device, such that the proximal part 11 of the sheath has a shape roughly of a truncated cone.

### Distal Part

The inner surface and the outer surface of the proximal part 11 of the balanic sheath are in material continuity with the inner surface and the outer surface of the distal part 12 of the sheath.

The inner surface of the distal part 12 of the sheath is preferably funnel-shaped. The outer surface has for its part a protuberance B described in more detail below.

The inner surface of the distal part 12 can also have several narrowed zones, down to a minimum narrowing of smaller diameter of between 0.5 and 3 mm, preferably of between 1 and 4 mm, more preferably 3 mm, at the distal end of the distal part 12.

The distal end of the distal part 12 of the balanic sheath is intended to collect the urine and direct it toward the outside of the sheath, for example by being connected to tubing, and then to a bag, in particular via a connection part 13. This connection part 13 can be a tube formed integrally with the distal part 12 or added to the latter. The outer diameter of the tube can be between 4 and 8 mm, in particular between 5 and 7 mm, for example 6 mm. The internal diameter of the tube can be between 1.5 and 3.5 mm, in particular between 2 and 3 mm, for example 2.5 mm.

### The Protuberance

The balanic sheath 10 of the urine drainage device of the present invention is characterized in that the outer surface of the distal part 12 of the sheath has an outwards protuberance B provided at a predetermined distance d from the proximal end of large diameter Dp of the sheath, such that the fastening system tightens the patient's foreskin upstream of the protuberance B, on a part C straddling the proximal part and the distal part 12, upstream being the part closest to the proximal end of large diameter, and downstream being the part closest to the small-diameter end of the sheath.

The protuberance B makes it possible to fasten the foreskin upstream of the latter by virtue of the fastening system. The protuberance B makes it possible, for example, to ensure that the fastening system does not slide along the sheath. Depending on the predetermined distance d, it is possible for the patient to have a balanic sheath 10 perfectly adapted to his morphology. A patient having longer than average foreskin can thus choose a sheath whose predetermined distance d is longer than average.

The protuberance B is for example a “hump” located on the outer surface of the distal part of the sheath. The hump is a volume like a half-ball characterized by a center and a diameter. In this case, the predetermined distance d is the distance between the center of the half-ball and the proximal end of the sheath, projected onto the height of the truncated cone of the sheath (according to the longitudinal axis of the truncated cone therefore). In addition to the predetermined distance d, the diameter I of the half-ball can also be adjusted, depending for example on the fastening system implemented.

According to one embodiment of the present invention, the protuberance is a continuous or non-continuous circular bead B, provided on a disk perpendicularly intersecting the distal part of the balanic sheath when the latter is seen from the side with the longitudinal axis of the cone collinear with a vertical axis.

A discontinuous bead B can, for example, increase the breathability of the foreskin. A continuous bead B is able to optimize the fixation of the sheath on the foreskin with the fastening system. A compromise between these two effects can be chosen.

Reference is now made to FIG. 2 which shows a sectional view of the urine drainage device according to the invention when the longitudinal axis of the balanic sheath is collinear with a vertical axis according to an embodiment where the protuberance B is a continuous bead.

Seen in cross section, the bead B may for example correspond to a so-called bell curve or a Gaussian curve or a parabola. A person skilled in the art is able to choose a curve that allows an optimum fixation of the fastening system upstream of the bead.

The bead B is defined by its thickness e, its height I, and its predetermined distance d from the proximal end of large diameter of the balanic sheath. The bead can thus be characterized by a height I of between 5 and 20 mm, preferably between 5 and 15 mm, more preferably 9 mm; a thickness e of the bead between the inner surface and the outer surface can be between 3 and 20 mm, preferably between 5 and 15 mm, more preferably 7 mm, and a distance d from the proximal end of large diameter of the balanic sheath of between 15 and 25 mm, preferably 21 mm.

The thickness e is the thickness between the inner surface and the outer surface of the distal part of the balanic sheath forming the bead. This thickness can be solid (a single material forming the sheath for example) or this thickness can be filled with air, for example to lighten the weight of the sheath. Moreover, the outer surface of the bead can be rigid or non-rigid. In the case where the bead is non-rigid, the user can for example deform it by pressing it. This can, for example, make it easier to fit a particular fastening system.

Reference is now made to FIG. 3 which shows a perspective view of the sheath according to an embodiment of the present invention where the protuberance B is a continuous bead. FIG. 3 shows the proximal end of large diameter Dp intended to be placed on the glans of the patient's penis, as well as the bead B placed at a predetermined distance from the proximal end Dp. Between the bead B and the proximal end Dp, the balanic sheath has a hollow C straddling the proximal part and the distal part. Once placed on the glans of the penis, the patient's foreskin is rolled over the outer surface of the proximal part of the sheath (upstream of the protuberance B which is a bead in this embodiment). The foreskin then is housed in the hollow C formed upstream of the bead B.

The hollow C is the result of the frustoconical shape of the inner surface of the proximal part 11 of the balanic sheath and the position of the protuberance straddling the proximal part 11 and the distal part 12. It is in this hollow that the foreskin is housed and the fastening system holds the balanic sheath 10 on the glans of the patient's penis.

### The Fastening System

Several fastening systems have been devised by the inventors to hold the patient's foreskin on the balanic sheath of the urine drainage device of the present invention.

### Non-Rigid Tightening Collar

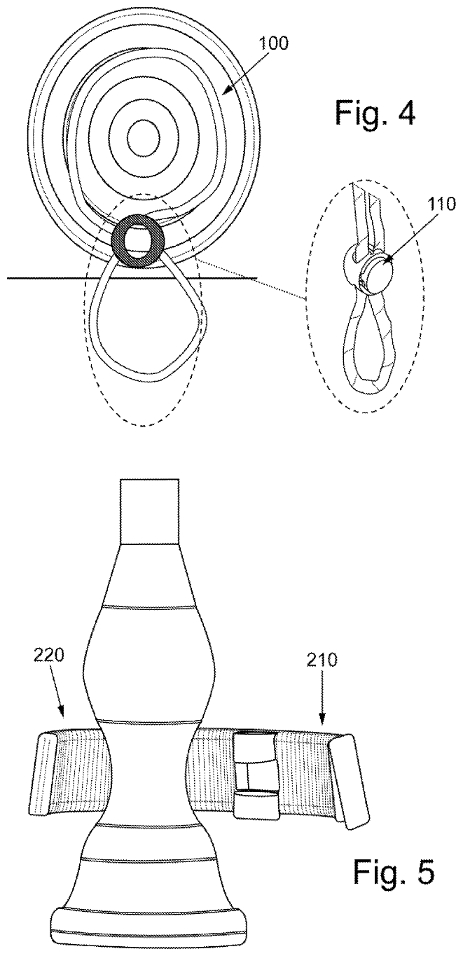

Reference is made to FIG. 4 which shows a front view through the distal end of small diameter of the balanic sheath of the urine drainage device according to an embodiment of the present invention in which the fastening system 100 is a non-rigid tightening collar in the form of a cord which is tightened using a tightening button 110 of the cord lock type. However, the non-rigid tightening collar can also be, for example, a touch-and-close band, or a cord which closes using a pushbutton, or an elastic circular band, or a flexible magnet in the form of a band, or a cord (in particular elastic) which closes by mean of a magnet. In order to ensure adaptability in the case of a cord which closes by mean of a pushbutton, the cord may be elastic or have a plurality of female pushbutton parts along the cord and a single male pushbutton part at the end of the cord, or alternatively a plurality of male pushbutton parts along the cord and a single female pushbutton part at the end of the cord.

Once the patient's foreskin has been unrolled on the outer surface of the balanic sheath, upstream of the protuberance, the user places the cord upstream of the protuberance and tightens it using the tightening button of the cord lock type 110. The tightening button of the cord lock type makes it possible to easily adjust the tightening throughout the day and to adapt to the different morphologies of the penises of patients.

The cord 100 is fully visible in FIG. 4 in order to facilitate understanding of the invention. In reality, the part of the cord that is tightened around the foreskin is hidden at least in part by the protuberance when the balanic sheath is seen from the front through the distal end of small diameter.

### Rigid Tightening Collar

Reference is now made to FIG. 5 which is a side view of the urine drainage device of the present invention when the longitudinal axis of the balanic sheath is collinear with a vertical axis, according to an embodiment in which the fastening system is a rigid tightening collar comprising two articulated parts 210 and 220 which are closed around the foreskin by mean of a hook-and-loop band or a pin-type fastening system.

This fastening system is practical to implement and it can be easily washed or sterilized. It also has a long lifespan.

### Clip System

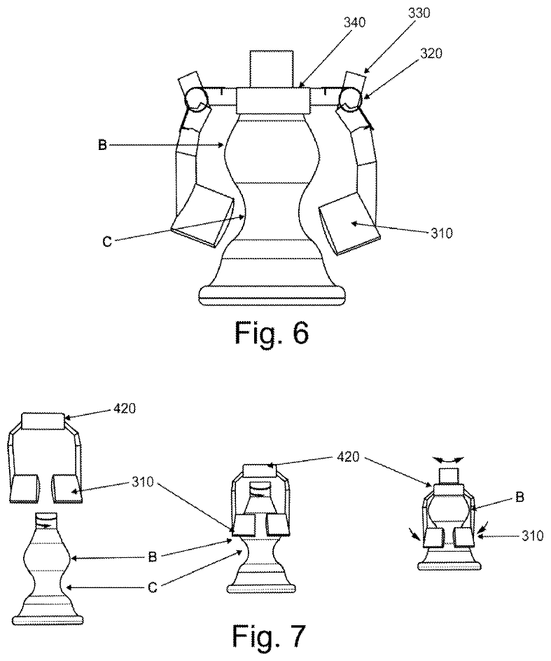

Reference is now made to FIG. 6 which shows a side view of the urine drainage device when the longitudinal axis of the balanic sheath is collinear with a vertical axis, according to an embodiment in which the fastening system is a clip system, of which the clips 310 tighten the foreskin on the sheath upstream of the bead, and the operating mechanism 320 of the clips is located downstream of the bead, the fastening system being open.

FIG. 6 illustrates the embodiment in which the operating mechanism 320 of the clips is a spring-operated system. The fastening system can further comprise two protuberances 330, by mean of which the user can open the clips by exerting pressure on the protuberances 330. Once the pressure is released, the elastic force of the springs tightens the foreskin upstream of the bead B, at the hollow C where the foreskin unrolled over the sheath is present. By adjusting the elastic force of the springs, it is possible to adjust the tightening of the patient's foreskin on the sheath. The fastening system can be integral with the sheath, or it may be possible to exchange it. For example, the fastening system is glued, clipped or even screwed onto the balanic sheath by mean of a ring 340 placed downstream of the bead B.

The end of the clips 310 coming into contact with the foreskin in order to tighten the latter on the balanic sheath can comprise a magnet which fastens magnetically to another magnet then provided in or on the sheath. The magnets can replace or reinforce the operating mechanism of the clips using a spring 320 for example.

Reference is now made to FIG. 7 which shows another embodiment in which the operating mechanism of the clips is no longer a spring-operated mechanism or one comprising protuberances on which the user can press, but a ring 420 which screws onto a thread provided downstream of the bead or clips onto a notch provided downstream of the bead, such that the clips 310 are tightened on the patient's foreskin upstream of the bead B when the ring is screwed or clipped downstream of the bead.

Here again, the clips tighten the foreskin upstream of the bead B, at the hollow C present on the sheath, straddling the proximal part and the distal part.

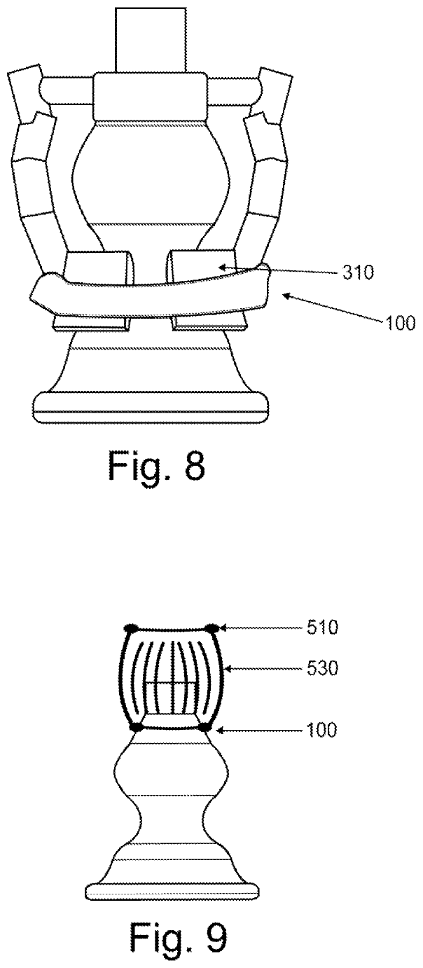

The two embodiments illustrated in FIGS. 6 and 7 can be combined with an embodiment in which the clips 310 are held tight around the foreskin by mean of the non-rigid tightening collar 100 as defined above and illustrated in FIG. 8.

### System of the “Skirt” Type

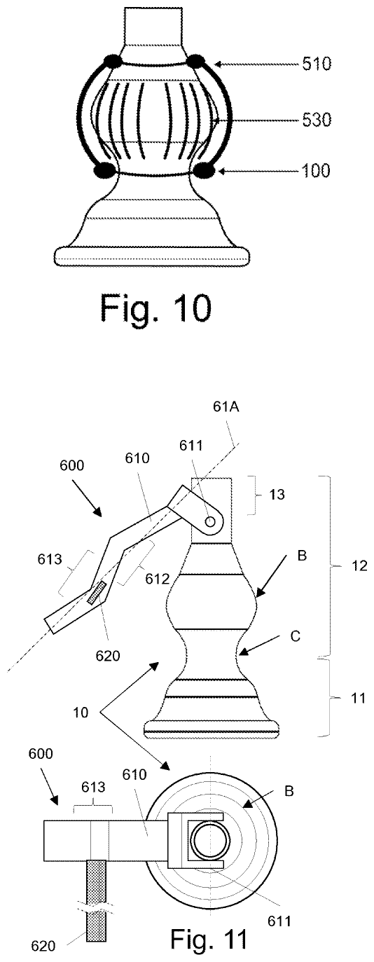

Reference is now made to FIGS. 9 and 10 which illustrate an embodiment of the present invention in which the fastening system is of the so-called “skirt” type and comprises a first circular elastic band 510 located downstream of the bead B and a non-rigid tightening collar 100 placed so as to tighten the patient's foreskin upstream of the bead B, the first elastic band 510 and the non-rigid tightening collar 100 are interconnected by a fine textile 530 such as silk or cotton or a silicone woven fabric.

The user fits the fastening system in place by sliding it over the balanic sheath and by pulling the elastic bands in order to enlarge them and place them on the sheath such that the first circular elastic band 510 tightens the sheath downstream of the bead B and the non-rigid tightening collar 100 tightens the foreskin upstream of the bead B.

### Non-Rigid Clamping Collar Coupled to a Lever

Reference is now made to FIGS. 11 to 13 which illustrate an embodiment in which the fastening system 600 is a non-rigid tightening collar 620, for example a tightening band, coupled to a lever 610.

The lever 610 is mounted rotatably on the balanic sheath, in particular on the distal end of the latter, such that the axis of rotation 611 is perpendicular to the longitudinal axis of the balanic sheath. Thus, the lever 610 extends from the surface of the balanic sheath, for example from the distal end of the latter, to a free end. The axis of rotation 611 is typically located below the connection part 13 for connection to a liquid evacuation tubing of the balanic sheath 10.

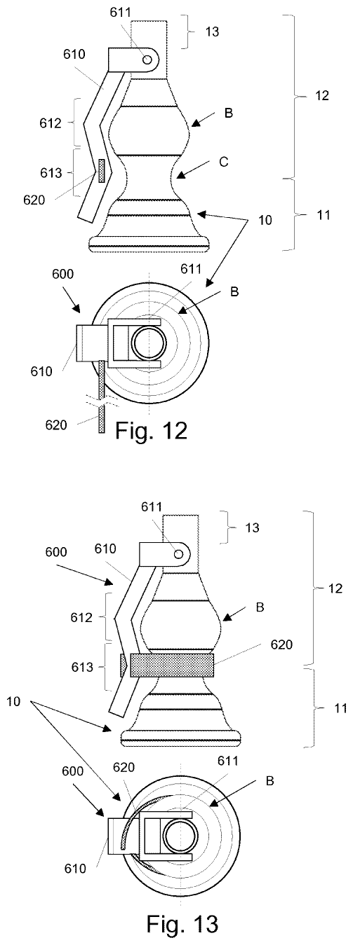

The lever 610 comprises a closed position (or even lowered position, see FIGS. 12, 13, 15 and 16), in which it is close to the balanic sheath and extends along the latter. In particular, it is in this position that its free end is closest to the surface of the balanic sheath. The shape of the lever is chosen such that the lever, in this closed position, is as close as possible to the surface of the balanic sheath. Thus, the lever comprises, along its extent from its attachment point 611, a projecting portion 612 which deviates from the longitudinal axis of the balanic sheath (corresponding to the protuberance), and a recessed portion 613 which approaches the longitudinal axis of the balanic sheath (corresponding to the zone straddling the proximal part and the distal part).

Still in this position, the recessed portion 613 forms, with the straddling zone C, a gap preferably of 0.5 to 5 mm. This gap corresponds to the thickness of the foreskin. In general, the lever 610 and the balanic sheath together form a gap of between 0.5 and 5 mm along the entire length of the balanic sheath. Preferably, this gap is constant all along the balanic sheath.

The lever 610 also has a maximum open position (or fully raised, see FIGS. 11 and 14), in which its free end is furthest from the surface of the balanic sheath. The maximum opening can be obtained when the middle axis 61A of the lever 610 and the longitudinal axis of the balanic sheath form an angle of 10 to 80°.

The material for the lever 610 can be one of the materials mentioned above for the balanic sheath. Preferably, the material of the lever and the material of the balanic sheath are identical.

The non-rigid tightening collar 620 is fixed to the lever 610, and in particular to its recessed portion 613, by one of its ends, and its other end is free. The non-rigid tightening collar 620 can be elastic or non-elastic. The non-rigid tightening collar 620 is adapted to close on itself after having surrounded the balanic sheath (see FIGS. 13 and 16).

Preferably, the non-rigid tightening collar 620 has the shape of a band, in particular with a length to width ratio of greater than 5. The width is preferably from 2 to 10 mm.

During use, having placed the end of the glans in the proximal part of the balanic sheath and unrolled the foreskin on the outer surface of the proximal part as far as the zone C straddling the proximal part and the distal part, the user lowers the lever 610 to the closed position, grasps the non-rigid tightening collar 620, which is then located opposite the straddle zone C, and passes it all round the straddle zone C of the balanic sheath in order to surround the latter and fasten the free end of the non-rigid tightening collar 620.

The fastening can be carried out in several ways: either fastening of the non-rigid tightening collar 620 on itself, or fastening of the non-rigid clamping collar 620 on the lever 610. In the case where the non-rigid tightening collar 620 is fixed on itself, it can comprise a touch-and-close system or pushbuttons. In the case where the non-rigid tightening collar 620 is fastened on the lever 610, the fastening can be a hook-loop or hook-hook system; in which case, the non-rigid tightening collar is preferably elastic.

When the user wishes to remove the system, he undoes the non-rigid tightening collar 620 and then raises the lever 610. It is not necessary to raise the lever 610 to its maximum open position; a few degrees of rotation may be enough to release the foreskin.

### Kit

According to a final aspect, the invention relates to a kit which comprises the urine drainage device described above, tubing, and a urine collection bag. The tubing can be fastened to the distal end of small diameter of the balanic sheath by being screwed, glued or built in. Moreover, the tubing and the balanic sheath may be integral and made in the same mold.

1-18. (canceled)

19. A urine drainage device comprising a non-adhesive balanic sheath for urine discharge and a fastening system,
wherein the balanic sheath comprises a proximal part for receiving the exposed glans of a patient, the proximal part of the balanic sheath having a frustoconical inner surface and an outer surface, the inner surface and the outer surface each comprising a distal end with a small diameter and a proximal end with a large diameter,
wherein the fastening system is configured to hold the patient's foreskin unrolled on the outer surface of the proximal part of the balanic sheath,
wherein the balanic sheath comprises a distal part continuing the proximal part, with an outer surface having a protuberance such that the foreskin, when it is unrolled, is held by the fastening system in a zone straddling the proximal part and the distal part.

20. The urine drainage device as claimed in claim 19, wherein the protuberance is a continuous or non-continuous circular bead, provided on a disk perpendicularly intersecting the distal part of the sheath, when the latter is viewed from the side with the axis of the cone collinear with a vertical axis.

21. The urine drainage device as claimed in claim 19, wherein the fastening system is a non-rigid, adaptable, tightening collar.

22. The urine drainage device as claimed in claim 19, wherein the fastening system is a rigid tightening collar comprising two articulated parts which are closed around the foreskin.

23. The urine drainage device as claimed in claim 19, wherein the fastening system is a clip system, of which the clips tighten the foreskin on the balanic sheath upstream of the protuberance, straddling the proximal part and the distal part of the sheath, and the operating mechanism of the clips is located downstream of the protuberance, at the distal part of the sheath.

24. The urine drainage device as claimed in claim 23, wherein the operating mechanism of the clips is a spring-operated system.

25. The urine drainage device as claimed in claim 23, wherein the end of the clips coming into contact with the foreskin in order to tighten the latter on the balanic sheath comprises a magnet which fastens magnetically to another magnet provided in or on the sheath.

26. The urine drainage device as claimed in claim 23, wherein the operating mechanism of the clips is a ring which screws onto a thread provided on the distal part of the balanic sheath or clips into a notch provided on the distal part of the balanic sheath, such that the clips tighten onto the patient's foreskin upstream of the protuberance when the ring is screwed or clipped on downstream of the protuberance.

27. The urine drainage device as claimed in claim 23, wherein the fastening system is a non-rigid, adaptable, tightening collar, and wherein the clips are held tightened around the foreskin by mean of the non-rigid tightening collar.

28. The urine drainage device as claimed in claim 23, wherein the fastening system is a non-rigid, adaptable, tightening collar, and wherein the fastening system is of the “skirt” type comprising a first circular elastic band downstream of the protuberance and the non-rigid tightening collar placed so as to tighten the patient's foreskin upstream of the protuberance, straddling the distal part and the proximal part of the balanic sheath, the first elastic band and the non-rigid tightening collar being connected to each other by a fine textile such as silk or cotton or a silicone weave.

29. The urine drainage device as claimed in claim 21, further comprising a lever mounted rotatably on the balanic sheath such that the axis of rotation is perpendicular to the longitudinal axis of the balanic sheath, and having a closed position in which it extends along the balanic sheath,
wherein the non-rigid tightening collar is fixed to the lever and adapted to close on itself and surround the balanic sheath.

30. The urine drainage device as claimed in claim 29, wherein the lever comprises, along its extent from its point of attachment to the balanic sheath, a projecting portion deviating from the longitudinal axis of the balanic sheath and corresponding to the protuberance, and a recessed portion approaching the longitudinal axis of the balanic sheath and corresponding to the zone straddling the proximal part and the distal part.

31. The urine drainage device as claimed in claim 30, wherein the non-rigid tightening collar is fastened to the recessed portion of the lever.

32. The urine drainage device as claimed in claim 19, wherein the balanic sheath is made of a material chosen from the group consisting of polyvinyl chloride, silicones, polyethylene, polystyrene, polypropylene, polyethylene terephthalate, polyacrylate, polymethyl acrylate and polyurethane, and the outer surface of the balanic sheath is made of a material chosen from the group consisting of polyvinyl chloride, silicones, polyethylene, polystyrene, polypropylene, polyethylene terephthalate, polyacrylate, polymethyl acrylate and polyurethane.

33. The urine drainage device as claimed in claim 19, wherein the balanic sheath is washable and sterilizable.

34. The urine drainage device as claimed in claim 20, wherein the protuberance is a bead which, seen from the side when the longitudinal axis of the balanic sheath is collinear with a vertical axis, has a shape according to a bell curve or Gaussian curve, the bead has a height of between 5 and 20 mm and a thickness between the inner surface and outer surface of between 3 and 20 mm.

35. The urine drainage device as claimed in claim 19, wherein the length of the proximal part of the balanic sheath having a frustoconical inner surface is between 5 and 25 mm and the ratio between the large diameter of the inner surface of the proximal part of the balanic sheath and the smaller diameter of the distal end of the proximal part of the balanic sheath is between 1 and 3.

36. A kit comprising a urine drainage device, tubing, and a urine collection bag,
wherein the urine drainage device comprises a non-adhesive balanic sheath for urine discharge and a fastening system,
the balanic sheath comprising a proximal part for receiving the exposed glans of a patient, the proximal part of the balanic sheath having a frustoconical inner surface and an outer surface, the inner surface and the outer surface each comprising a distal end with a small diameter and a proximal end with a large diameter,
the fastening system holding the patient's foreskin unrolled on the outer surface of the proximal part of the balanic sheath,
wherein the balanic sheath comprises a distal part continuing the proximal part, with an outer surface having a protuberance such that the foreskin, when it is unrolled, is held by the fastening system in a zone straddling the proximal part and the distal part.
